SEQUENCE LISTING (10 sequences)

SEQ ID NO: 1 - protein, 11 aa, Artificial Sequence
  Description of Artificial Sequence VSV-tag
YTDIEMNRLGK

SEQ ID NO: 2 - DNA, 3561 bp, Artificial Sequence
  Description of Artificial Sequence Phagemid PDV UO3
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtctaaatgttgaaaaagaaaaacatttattcaattcgtaaattaggtgtaggtattgcatctgtaacgttaggtaccttacttatctctggtggcgtaacaccggctgcaaatgcttccatgggctatccgtacgacgttccggattatgcctaactcgagttatataccgatattgaaatgaaccgcctgggcaaaggcggtcgtgccagccgcttaaaaggcgtgagcaccccgccgagcccgcagttaattaacgctgagggtgacgatcccgcaaaagcggcctttgactccctgcaagcctcagcgaccgaatatatcggttatgcgtgggcgatggttgttgtcattgtcggcgcaactatcggtatcaagctgtttaagaaattcacctcgaaagcaagctgattaattaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatataaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 3 - DNA, 3561 bp, Artificial Sequence
  Description of Artificial Sequence Phagemid PDV UO3
cgcgggttatgcgtttggcggagaggggcgcgcaaccggctaagtaattacgtcgaccgtgctgtccaaagggctgacctttcgcccgtcactcgcgttgcgttaattacactcaatcgagtgagtaatccgtggggtccgaaatgtgaaatacgaaggccgagcatacaacacaccttaacactcgcctattgttaaagtgtgtcctttgtcgatactggtactaatgcggttcgaacgtacgtttaagataaagttcctctgtcagatttacaactttttctttttgtaaataagttaagcatttaatccacatccataacgtagacattgcaatccatggaatgaatagagaccaccgcattgtggccgacgtttacgaaggtacccgataggcatgctgcaaggcctaatacggattgagctcaatatatggctataactttacttggcggacccgtttccgccagcacggtcggcgaattttccgcactcgtggggcggctcgggcgtcaattaattgcgactcccactgctagggcgttttcgccggaaactgagggacgttcggagtcgctggcttatatagccaatacgcacccgctaccaacaacagtaacagccgcgttgatagccatagttcgacaaattctttaagtggagctttcgttcgactaattaattcttaagtgaccggcagcaaaatgttgcagcactgacccttttgggaccgcaatgggttgaattagcggaacgtcgtgtagggggaaagcggtcgaccgcattatcgcttctccgggcgtggctagcgggaagggttgtcaacgcgtcggacttaccgcttaccgcggactacgccataaaagaggaatgcgtagacacgccataaagtgtggcgtatatttaacatttgcaattataaaacaattttaagcgcaatttaaaaacaatttagtcgagtaaaaaattggttatccggctttagccgttttagggaatatttagttttcttatcgggctctatcccaactcacaacaaggtcaaaccttgttctcaggtgataatttcttgcacctgaggttgcagtttcccgctttttggcagatagtcccgctaccgggtgatgcacttggtagtgggtttagttcaaaaaaccccagctccacggcatttcgtgatttagccttgggatttccctcgggggctaaatctcgaactgcccctttcggccgcttgcaccgctctttccttcccttctttcgctttcctcgcccgcgatcccgcgaccgttcacatcgccagtgcgacgcgcattggtggtgtgggcggcgcgaattacgcggcgatgtcccgcgcatgataccaacgaaactgcccacgtgagagtcatgttagacgagactacggcgtatcaattcggtcggggctgtgggcggttgtgggcgactgcgcgggactgcccgaacagacgagggccgtaggcgaatgtctgttcgacactggcagaggccctcgacgtacacagtctccaaaagtggcagtagtggctttgcgcgctctgctttcccggagcactatgcggataaaaatatccaattacagtactattattaccaaagaatctgcagtccaccgtgaaaagcccctttacacgcgccttggggataaacaaataaaaagatttatgtaagtttatacataggcgagtactctgttattgggactatttacgaagttattataactttttccttctcatactcataagttgtaaaggcacagcgggaataagggaaaaaacgccgtaaaacggaaggacaaaaacgagtgggtctttgcgaccactttcattttctacgacttctagtcaacccacgtgctcacccaatgtagcttgacctagagttgtcgccattctaggaactctcaaaagcggggcttcttgcaaaaggttactactcgtgaaaatttcaagacgatacaccgcgccataatagggcataactgcggcccgttctcgttgagccagcggcgtatgtgataagagtcttactgaaccaactcatgagtggtcagtgtcttttcgtagaatgcctaccgtactgtcattctcttaatacgtcacgacggtattggtactcactattgtgacgccggttgaatgaagactgttgctagcctcctggcttcctcgattggcgaaaaaacgtgttgtaccccctagtacattgagcggaactagcaacccttggcctcgacttacttcggtatggtttgctgctcgcactgtggtgctacggacatcgttaccgttgttgcaacgcgtttgataattgaccgcttgatgaatgagatcgaagggccgttgttaattatctgacctacctccgcctatttcaacgtcctggtgaagacgcgagccgggaaggccgaccgaccaaataacgactatttagacctcggccactcgcacccagagcgccatagtaacgtcgtgaccccggtctaccattcgggagggcatagcatcaatagatgtgctgcccctcagtccgttgatacctacttgctttatctgtctagcgactctatccacggagtgactaattcgtaaccattgacagtctggttcaaatgagtatatatgaaatctaactaaattttgaagtaaaaattaaattttcctagatccacttctaggaaaaactattagagtactggttttagggaattgcactcaaaagcaaggtgactcgcagtctggggcatcttttctagtttcctagaagaactctaggaaaaaaagacgcgcattagacgacgaacgtttgtttttttggtggcgatggtcgccaccaaacaaacggcctagttctcgatggttgagaaaaaggcttccattgaccgaagtcgtctcgcgtctatggtttatgacaggaagatcacatcggcatcaatccggtggtgaagttcttgagacatcgtggcggatgtatggagcgagacgattaggacaatggtcaccgacgacggtcaccgctattcagcacagaatggcccaacctgagttctgctatcaatggcctattccgcgtcgccagcccgacttgccccccaagcacgtgtgtcgggtcgaacctcgcttgctggatgtggcttgactctatggatgtcgcactcgatactctttcgcggtgcgaagggcttccctctttccgcctgtccataggccattcgccgtcccagccttgtcctctcgcgtgctccctcgaaggtccccctttgcggaccatagaaatatcaggacagcccaaagcggtggagactgaactcgcagctaaaaacactacgagcagtccccccgcctcggatacctttttgcggtcgttgcgccggaaaaatgccaaggaccggaaaacgaccggaaaacgagtgtacaagaaaggacgcaataggggactaagacacctattggcataatggcggaaactcactcgactatggcgagcggcgtcggcttgctggctcgcgtcgctcagtcactcgctccttcgccttct

SEQ ID NO: 4 - protein, 96 aa, Artificial Sequence
  Description of Artificial Sequence Soluble VH3 domain core
QVQLVESGGGLVQAGGSLRLSCAASGXXXXXXYMGWFRQAPGKERELVAAIXXGXSTYYADSVKGRFTISRDNAKNTVYLQMNSLKPEDTAVYYCA

SEQ ID NO: 5 - DNA, 378 bp, Artificial Sequence
  Description of Artificial Sequence PDV-UO2 cloning region
tgcatgcaaattctatttcaaggagacagtctaaatgttgaaaaagaaaaacatttattcaattcgtaaattaggtgtaggtattgcatctgtaacgttaggtaccttacttatctctggtggcgtaacaccggctgcaaatgcttccatgggctatccgtacgacgttccggattatgcctaactcgagggtaccggaggttccggcggaaccgggtctgggactggtacgagcgagctcgaacagaaattaatctctgaggaagacttggcggccgcattatataccgatattgaaatgaaccgcctgggcaaaggctagggtcgtgccagccgcttaaaaggcgtgagcaccccgccgagcccgcagttaattaa

SEQ ID NO: 6 - DNA, 378 bp, Artificial Sequence
  Description of Artificial Sequence PDV-UO2 cloning region
acgtacgtttaagataaagttcctctgtcagatttacaactttttctttttgtaaataagttaagcatttaatccacatccataacgtagacattgcaatccatggaatgaatagagaccaccgcattgtggccgacgtttacgaaggtacccgataggcatgctgcaaggcctaatacggattgagctcccatggcctccaaggccgccttggcccagaccctgaccatgctcgctcgagcttgtctttaattagagactccttctgaaccgccggcgtaatatatggctataactttacttggcggacccgtttccgatcccagcacggtcggcgaattttccgcactcgtggggcggctcgggcgtcaattaatt

SEQ ID NO: 7 - DNA, 4715 bp, Artificial Sequence
  Description of Artificial Sequence PDV-UO2 sequence
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtctaaatgttgaaaaagaaaaacatttattcaattcgtaaattaggtgtaggtattgcatctgtaacgttaggtaccttacttatctctggtggcgtaacaccggctgcaaatgcttccatgggctatccgtacgacgttccggattatgcctaactcgagggtaccggaggttccggcggaaccgggtctgggactggtacgagcgagctcgaacagaaattaatctctgagcaagacttggcggccgcattatataccgatattgaaatgaaccgcctgggcaaaggctagggtcgtgccagccgcttaaaaggcgtgagcaccccgccgagcccgcagttaattaacgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaattaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatataaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 8 - DNA, 4715 bp, Artificial Sequence
  Description of Artificial Sequence PDV U02 sequence
cgcgggttatgcgtttggcggagaggggcgcgcaaccggctaagtaattacgtcgaccgtgctgtccaaagggctgacctttcgcccgtcactcgcgttgcgttaattacactcaatcgagtgagtaatccgtggggtccgaaatgtgaaatacgaaggccgagcatacaacacaccttaacactcgcctattgttaaagtgtgtcctttgtcgatactggtactaatgcggttcgaacgtacgtttaagataaagttcctctgtcagatttacaactttttctttttgtaaataagttaagcatttaatccacatccataacgtagacattgcaatccatggaatgaatagagaccaccgcattgtggccgacgtttacgaaggtacccgataggcatgctgcaaggcctaatacggattgagctcccatggcctccaaggccgccttggcccagaccctgaccatgctcgctcgagcttgtctttaattagagactccttctgaaccgccggcgtaatatatggctataactttacttggcggacccgtttccgatcccagcacggtcggcgaattttccgcactcgtggggcggctcgggcgtcaattaattgctttgacaactttcaacaaatcgttttggagtatgtcttttaagtaaatgattgcagacctttctgctgttttgaaatctagcaatgcgattgatactcccgacagacaccttacgatgtccgcaacaccaaacatgaccactgctttgagtcacaatgccatgtacccaaggataacccgaacgatagggacttttactcccaccaccgagactcccaccgccaagactcccaccgccaagactcccaccgccatgatttggaggactcatgccactatgtggataaggcccgatatgaatatagttgggagagctgccgtgaataggcggaccatgactcgttttggggcgattaggattaggaagagaactcctcagagtcggagaattatgaaagtacaaagtcttattatccaaggctttatccgtcccacgtaattgacaaatatgcccgtgacaatgagttccgtgactggggcaattttgaataatggtcatgtgaggacatagtagttttcggtacatactgcgaatgaccttgccatttaagtctctgacgcgaaaggtaagaccgaaattactcctaggtaagcaaacacttatagttccggttagcagactggacggagttggaggacagttacgaccgccgccgagaccaccaccaagaccaccgccgagactcccaccgccgagactcccaccgccaagactcccaccgccgagactcccaccgccaaggccaccgccgaggccaaggccactaaaactaatacttttttaccgtttgcgattattcccccgatactggcttttacggctacttttgcgcgatgtcagactgcgatttccgtttgaactaagacagcgatgactaatgccacgacgatagctaccaaagtaaccactgcaaaggccggaacgattaccattaccacgatgaccactaaaacgaccgagattaagggtttaccgagttcagccactgccactattaagtggaaattacttattaaaggcagttataaatggaagaaacggagtcagccaacttacagcgggaatacagaaaccgcgaccatttggtatacttaaaagataactaacactgttttatttgaataaggcaccacagaaacgcaaagaaaatatacaacggtggaaatacatacataaaagctgcaaacgattgtatgacgcattattcctcagaattaattcttaagtgaccggcagcaaaatgttgcagcactgacccttttgggaccgcaatgggttgaattagcggaacgtcgtgtagggggaaagcggtcgaccgcattatcgcttctccgggcgtggctagcgggaagggttgtcaacgcgtcggacttaccgcttaccgcggactacgccataaaagaggaatgcgtagacacgccataaagtgtggcgtatatttaacatttgcaattataaaacaattttaagcgcaatttaaaaacaatttagtcgagtaaaaaattggttatccggctttagccgttttagggaatatttagttttcttatcgggctctatcccaactcacaacaaggtcaaaccttgttctcaggtgataatttcttgcacctgaggttgcagtttcccgctttttggcagatagtcccgctaccgggtgatgcacttggtagtgggtttagttcaaaaaaccccagctccacggcatttcgtgatttagccttgggatttccctcgggggctaaatctcgaactgcccctttcggccgcttgcaccgctctttccttcccttctttcgctttcctcgcccgcgatcccgcgaccgttcacatcgccagtgcgacgcgcattggtggtgtgggcggcgcgaattacgcggcgatgtcccgcgcatgataccaacgaaactgcccacgtgagagtcatgttagacgagactacggcgtatcaattcggtcggggctgtgggcggttgtgggcgactgcgcgggactgcccgaacagacgagggccgtaggcgaatgtctgttcgacactggcagaggccctcgacgtacacagtctccaaaagtggcagtagtggctttgcgcgctctgctttcccggagcactatgcggataaaaatatccaattacagtactattattaccaaagaatctgcagtccaccgtgaaaagcccctttacacgcgccttggggataaacaaataaaaagatttatgtaagtttatacataggcgagtactctgttattgggactatttacgaagttattataactttttccttctcatactcataagttgtaaaggcacagcgggaataagggaaaaaacgccgtaaaacggaaggacaaaaacgagtgggtctttgcgaccactttcattttctacgacttctagtcaacccacgtgctcacccaatgtagcttgacctagagttgtcgccattctaggaactctcaaaagcggggcttcttgcaaaaggttactactcgtgaaaatttcaagacgatacaccgcgccataatagggcataactgcggcccgttctcgttgagccagcggcgtatgtgataagagtcttactgaaccaactcatgagtggtcagtgtcttttcgtagaatgcctaccgtactgtcattctcttaatacgtcacgacggtattggtactcactattgtgacgccggttgaatgaagactgttgctagcctcctggcttcctcgattggcgaaaaaacgtgttgtaccccctagtacattgagcggaactagcaacccttggcctcgacttacttcggtatggtttgctgctcgcactgtggtgctacggacatcgttaccgttgttgcaacgcgtttgataattgaccgcttgatgaatgagatcgaagggccgttgttaattatctgacctacctccgcctatttcaacgtcctggtgaagacgcgagccgggaaggccgaccgaccaaataacgactatttagacctcggccactcgcacccagagcgccatagtaacgtcgtgaccccggtctaccattcgggagggcatagcatcaatagatgtgctgcccctcagtccgttgatacctacttgctttatctgtctagcgactctatccacggagtgactaattcgtaaccattgacagtctggttcaaatgagtatatatgaaatctaactaaattttgaagtaaaaattaaattttcctagatccacttctaggaaaaactattagagtactggttttagggaattgcactcaaaagcaaggtgactcgcagtctggggcatcttttctagtttcctagaagaactctaggaaaaaaagacgcgcattagacgacgaacgtttgtttttttggtggcgatggtcgccaccaaacaaacggcctagttctcgatggttgagaaaaaggcttccattgaccgaagtcgtctcgcgtctatggtttatgacaggaagatcacatcggcatcaatccggtggtgaagttcttgagacatcgtggcggatgtatggagcgagacgattaggacaatggtcaccgacgacggtcaccgctattcagcacagaatggcccaacctgagttctgctatcaatggcctattccgcgtcgccagcccgacttgccccccaagcacgtgtgtcgggtcgaacctcgcttgctggatgtggcttgactctatggatgtcgcactcgatactctttcgcggtgcgaagggcttccctctttccgcctgtccataggccattcgccgtcccagccttgtcctctcgcgtgctccctcgaaggtccccctttgcggaccatagaaatatcaggacagcccaaagcggtggagactgaactcgcagctaaaaacactacgagcagtccccccgcctcggatacctttttgcggtcgttgcgccggaaaaatgccaaggaccggaaaacgaccggaaaacgagtgtacaagaaaggacgcaataggggactaagacacctattggcataatggcggaaactcactcgactatggcgagcggcgtcggcttgctggctcgcgtcgctcagtcactcgctccttcgccttct

SEQ ID NO: 9 - protein, 98 aa, Artificial Sequence
  Description of Artificial Sequence Part of variable fragment
EVQLXESGGGLVXPGGSLRLSCAASGFTFSXXXMXWVRQAPGKGLEWVAXIXXDGXXXXYXDSVKGRFTISRDNXKNXLYLQMNSLRXEDTAXYYCAX

SEQ ID NO: 10 - DNA, 294 bp, Artificial Sequence
  Description of Artificial Sequence Nucleic Acid sequence encoding
gaagtgcagctgstggaaagcggcggcggcctggtgmagccgggcggcagcctgcgcctgagctgcgcagctagcggcttcaccttcagcrrckmckvsatgmvctgggtgcgccaggccccgggcaaaggcctcgagtgggtggccvwtattwrkbakgatggcmrcrawraatwttacgycgatagcgtgaaaggccgcttcaccatcagccgcgataackccaaaaacwccctgtacctgcagatgaacagcctgcgcgmcgaagataccgccstgtactactgcgcacgc

(19) United States
(12) Patent Application Publication
(10) Pub. No.: US 2003/0219829 A1
(43) Pub. Date: Nov. 27, 2003

(54) Heavy chain libraries

(76) Inventors: Ton Logtenberg, Werkhoven (NL); Erwin Houtzager, Amerongen (NL)

Correspondence Address: TRASK BRITT, P.O. BOX 2550, SALT LAKE CITY, UT, 84110, US

(21) Appl. No.: 10/382,361

(22) Filed: Mar. 5, 2003

Related U.S. Application Data
(63) Continuation of application No. PCT/NL01/00670, filed on Sep. 12, 2001.

(30) Foreign Application Priority Data
Mar. 20, 2002 (EP) EP 1 188 771
Sep. 15, 2000 (EP) 00203216.7

(51) Int. Cl. G01N 33/53; C07H 21/04; C12P 21/02; C12N 5/06; C07K 16/18

(52) U.S. Cl. 435/7.1; 435/69.1; 435/320.1; 435/326; 530/388.1; 536/23.53

(57) ABSTRACT

The invention provides libraries comprising binding molecules adapted to expression in an expression organism, but also transferable to a human context without undergoing a change in conformation and/or build up. A method for producing a human monoclonal antibody includes: providing a library of binding molecules, the binding domain of which consists essentially of human heavy chain variable fragments in a functional format, selecting from the library at least one heavy chain variable fragment having a desired binding affinity, and inserting a nucleic acid encoding the heavy chain variable fragment into a nucleic acid encoding the complementary part of at least a heavy chain of the human monoclonal antibody, allowing for expression of the resulting heavy chain and for assembly of the heavy chain with a desired light chain, and producing a human monoclonal antibody. The heavy chain variable fragment's conformation retains its binding affinity whether it is in phage display or in its normal heavy chain environment. A method for making a library for use in the method is also provided, as are methods of keeping heavy chain variable fragments in the conformation. The invention allows for the production of larger libraries than known ones. Further, loss of specificities and affinities due to expression problems are reduced.

### CROSS-REFERENCE TO RELATED APPLICATIONS

This application is a continuation of PCT International Patent Application No. PCT/NL/01/00670, filed on Sep. 12, 2001, designating the United States of America, and published, in English, as PCT International Publication No. WO 02/28903 A2 on Apr. 11, 2002 (see, also, European Patent Appln. EP 1 188 771 A1, published Mar. 20, 2002), the contents of the entirety of both which are incorporated by this reference. This application also claims benefit, under 35 USC §119(e), to U.S. Provisional Patent Appln. 60/232,192, filed on Sep. 13, 2002.

### TECHNICAL FIELD

The invention relates to the fields of molecular biology and immunology and, in particular, to the field of designing, for example, human antibodies having a desired binding affinity through display and selection techniques.

### BACKGROUND

The exposure to a highly diverse and continuously changing environment requires a dynamic immune system that is able to rapidly adapt in order to adequately respond to potentially harmful microorganisms. Higher organisms have evolved specialized molecular mechanisms to ensure the implementation of clonally-distributed, highly diverse repertoires of antigen-receptor molecules expressed by cells of the immune system: immunoglobulin (Ig) molecules on B lymphocytes and T cell receptors on T lymphocytes. For B lymphocytes, a primary repertoire of (generally low affinity) Ig receptors is established during B cell differentiation in the bone marrow as a result of rearrangement of germline-encoded gene segments. Further refinement of Ig receptor specificity and affinity takes place in peripheral lymphoid organs where antigen-stimulated B lymphocytes activate a somatic hypermutation machinery that specifically targets the immunoglobulin variable (V) regions. During this process, B cell clones with mutant Ig receptors of higher affinity for the inciting antigen are stimulated into clonal proliferation and maturation into antibody-secreting plasma cells (reviewed in 1).

Recently, recombinant DNA technology has been used to mimic many aspects of the processes that govern the generation and selection of natural human antibody repertoires (reviewed in 2, 3). For instance, the construction of large repertoires of antibody fragments expressed on the surface of filamentous phage particles and the selection of phages by “panning” on antigens has been developed as a versatile and rapid method to obtain antibodies of desired specificities (reviewed in 4,5). Further optimization of the affinity of individual phage antibodies has been achieved by creating mutant antibody repertoires that are expressed on bacteriophage particles and sampled for higher affinity mutants by selection for binding to antigen under stringent conditions (reviewed in 6). Various approaches have been used to create mutated antibody repertoires, including chain shuffling (7,8), error prone PCR (9), use of E. coli mutator strains (10) or approaches more specifically directed to the complementarity determining regions (“CDRs”) of the antibody molecule, like CDR “walking” and parsimonious mutagenesis (11-13).

Libraries created so far have a more limited span of specificities than possible. This is in large part due to the fact that many specificities present are not expressed or exposed properly by the organism, for example, chosen for expression of the library components. This is most likely due to a lack of adaptation of the expression products to the expression environment.

Furthermore, the libraries created so far, if they contain a desired specificity, require engineering of the nucleic acid encoding the specificity in order to be able to create a fully human monoclonal antibody. For instance, in single chain Fv molecules, the light chain encoding sequence and the heavy chain encoding sequence are separated from the linker sequence and separately inserted into a complementary part of a heavy chain encoding sequence and a light chain encoding sequence. Upon this rearranging of the variable parts, specificity and affinity may change.

The present invention solves these problems at least in part. Other advantages and embodiments of the present invention will be clear from the detailed description below.

### DISCLOSURE OF THE INVENTION

The invention now provides libraries which comprise binding molecules that are adapted to expression in the expression organism, but which are also transferable to a human context without undergoing a change in conformation and/or build up. Thus, the present invention provides a method for producing a human monoclonal antibody, said method comprising: providing a library of binding molecules, the binding domain of which consists essentially of human heavy chain variable fragments in a functional format, selecting from said library at least one heavy chain variable fragment having a desired binding affinity, and inserting a nucleic acid encoding said heavy chain variable fragment into a nucleic acid encoding the complementary part of at least a, heavy chain of said human monoclonal antibody, allowing for expression of the resulting heavy chain and for assembly of said heavy chain with a desired light chain, and producing a human monoclonal antibody. The present inventors have found that having only a heavy chain derived variable fragment determining the binding affinity of the binding molecules in the library, that, as long as they are presented in a functional format, this will suffice for creating a library at least as large as the known ones, but typically will allow for producing even larger libraries. Also, the loss of specificities and affinities because of expression problems can be reduced, especially according to the preferred embodiments as disclosed herein below. A heavy chain variable fragment is defined as anything based on a fragment the size of a CDR (complementarity determining region) of a heavy chain (e.g., CDR 3) to a heavy chain variable fragment as usually defined in the art. Also, the way the heavy chain variable fragments are encoded, allows for the direct insertion into a (preferably) standard complementary part of the heavy chain encoding nucleic acid without significantly altering its conformation, affinity and/or specificity. The resulting heavy chain (upon expression) can then be assembled with a (preferably standard) light chain. However, this light chain will typically not have any significant binding affinity for the molecule recognized by the heavy chain variable fragment.

The nucleic acids encoding the heavy and light chains of the resulting human monoclonal antibody may be the same or different. They typically are expressed in a eukaryotic cell, preferably a human cell, preferably a cell like PER.C6. It may be either transient expression or from insertions in the host cell's genome; the latter being preferred.

### DETAILED DESCRIPTION OF THE INVENTION

In a preferred embodiment, the methods of the invention are carried out in a manner wherein the heavy chain variable fragment is in a functional format through fusion to a structural protein designed for that purpose. A functional format means that its conformation is such that it retains it binding affinity whether it is in phage display, or in its normal heavy chain environment. Methods of keeping heavy chain variable fragments in such a conformation are an important aspect of the present invention. It is disclosed herein how to provide amino acid sequences capable of simulating the conformation of the heavy chain variable fragment in phage display surroundings the way they are in the natural surroundings. One way is fusing a variable fragment with a known affinity to random sequences, expressing the resulting nucleic acids and selecting for the known affinity. In another preferred embodiment, the equality of the conformation of the phage display fragment and the fragment in the heavy chain environment is removal of at least one sequence which is responsible for associating with a light chain. In this format, an indifferent light chain variable fragment can be used as a structural amino acid sequence. According to the invention, the heavy chain variable fragment is preferably inserted into a standard human heavy chain encoding nucleic acid, derived from a human antibody backbone which is prevalent in the population, these include, but are not limited to members of the VH1, VH3 or VH4 gene families. The same is true for the light chain. These include, but are not limited to members of the Vkappa1, Vkappa3 and Vlambda3 gene familes.

This way, the invention provides a kit of parts consisting of heavy chain variable fragments having the desired binding affinity to cut from the library and a set of ready to use monoclonal antibody encoding nucleic acids to insert them in.

Thus, the invention also provides a human monoclonal antibody obtainable by a method according to the invention as disclosed above. As explained previously herein, the invention provides a method for producing a structural amino acid sequence or a nucleic acid sequence encoding such an amino acid sequence for keeping a human heavy chain variable fragment in a functional format upon expression of a nucleic acid encoding such a fragment in a fusion with a nucleic acid encoding a protein expressed associated with the surface of a phage particle, comprising fusing a nucleic acid sequence encoding a possible structural amino acid sequence to a nucleic acid which is a fusion of a human heavy chain variable fragment with a known binding affinity and the nucleic acid encoding a protein expressed associated with the surface of a phage particle and expressing said nucleic acid in the context of a suitable phage expression system and selecting fusions which expose the desired binding affinity. The fusions in functional alignment basically mean that the order in which the sequences are present can be different and be functional. The heavy chain variable fragment and the structural amino acid sequence encoding parts should be next to each other, in either direction. The phage surface protein encoding nucleic acid can be on either side. The linkage may be direct or indirect. The amino acid sequence designed for keeping a heavy chain variable fragment in the proper conformation will work for other heavy chain variable fragments as well. The invention thus also includes these amino acid sequences (proteinaceous substances) and their encoding nucleic acids. Thus, one can make a library of heavy chain variable fragments in proper conformation, because of the presence of the novel structural sequence.

The invention further comprises a method for making a library for use in a method according to the invention, comprising cloning a number of randomized nucleic acids derived from a heavy chain variable fragment in functional alignment with a nucleic acid encoding a proteinaceous substance as disclosed hereinabove, and providing the resulting nucleic acid in functional alignment with a nucleic acid encoding a protein expressed associated with the surface of a phage particle and expressing the resulting nucleic acids comprising said heavy chain variable fragment, the proteinaceous substance encoding acid and said surface protein encoding nucleic acid in the context of a suitable phage expression system, thus producing said library. The invention also provides a phage display library obtainable by a method disclosed above.

### EXAMPLES

### Example 1

Generation of a library of heavy chain variable regions using a soluble variable heavy chain 3 domain (sVH3).

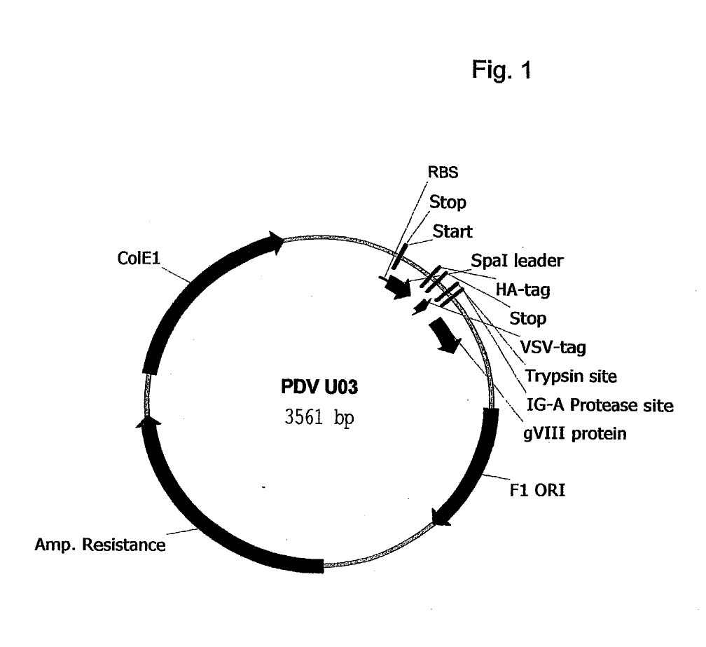

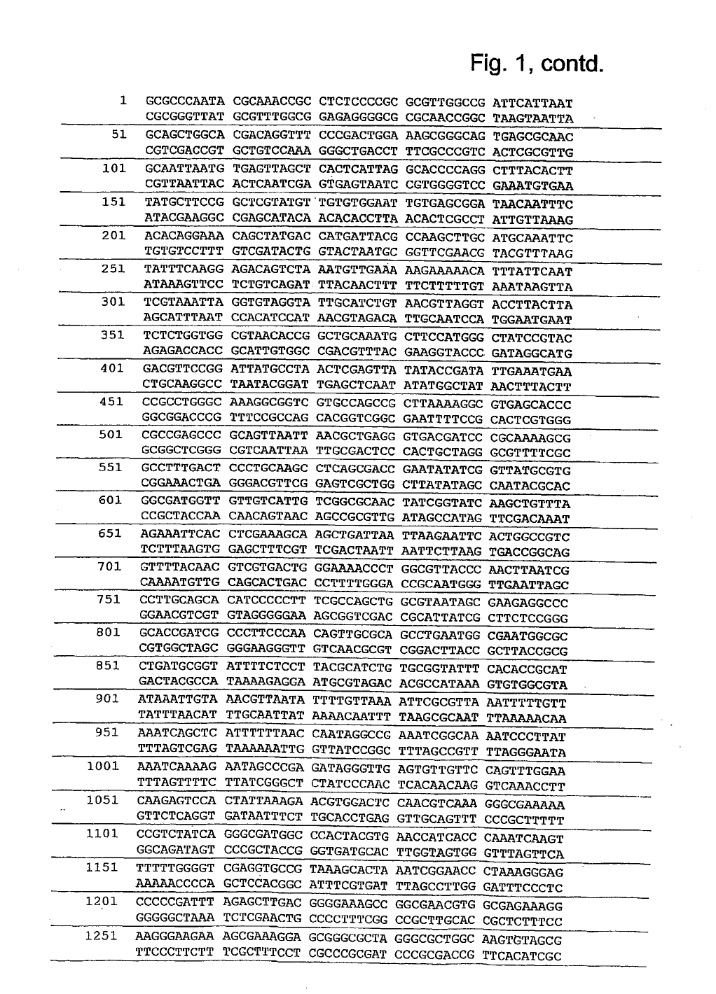

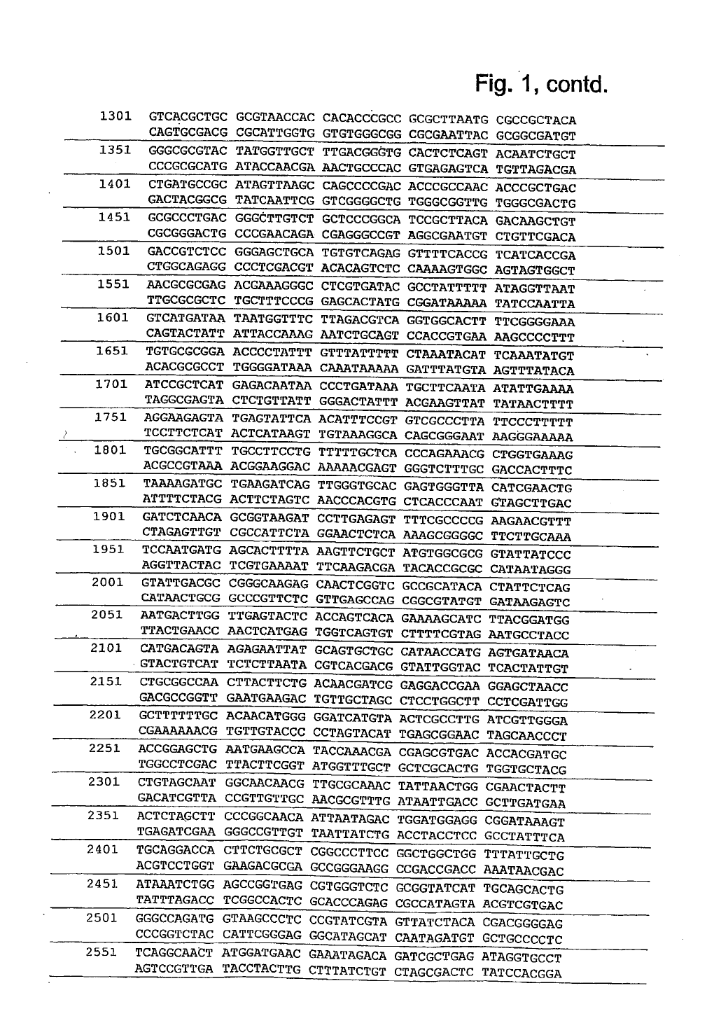

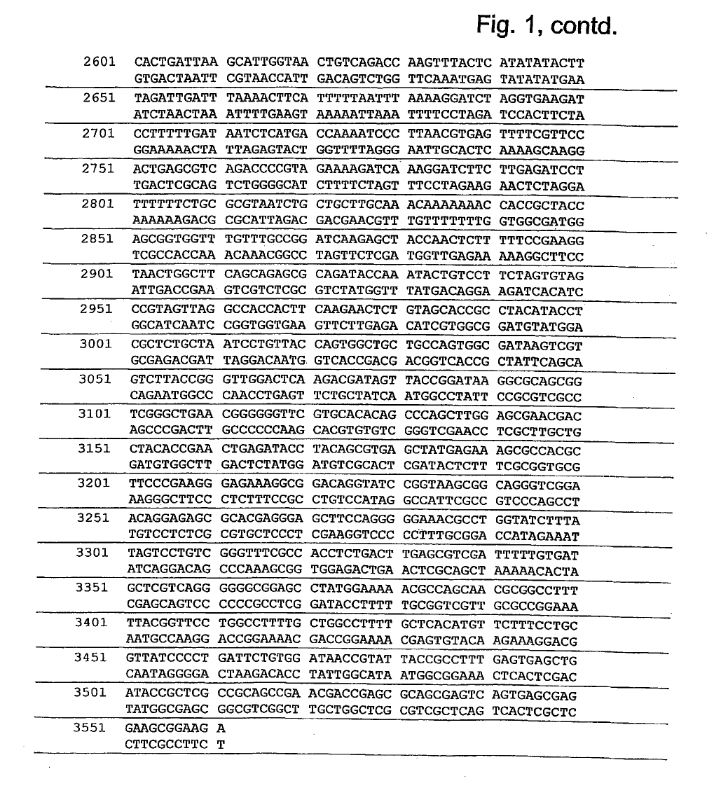

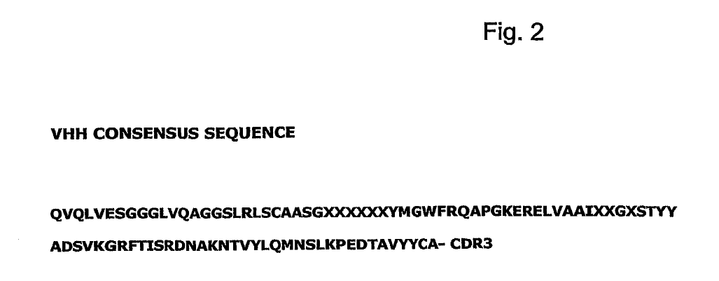

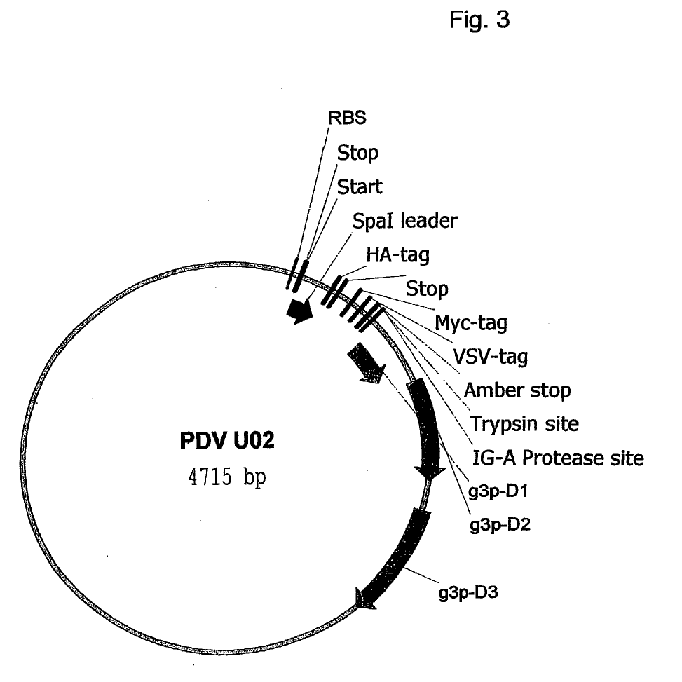

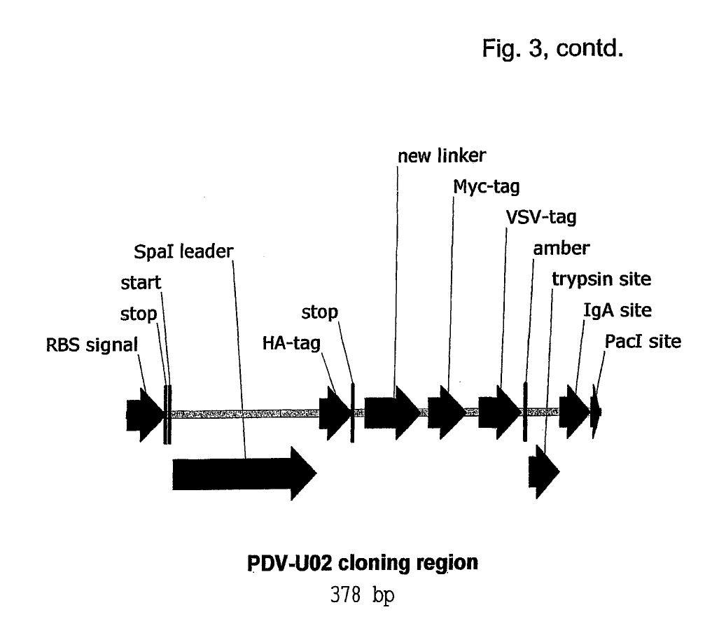

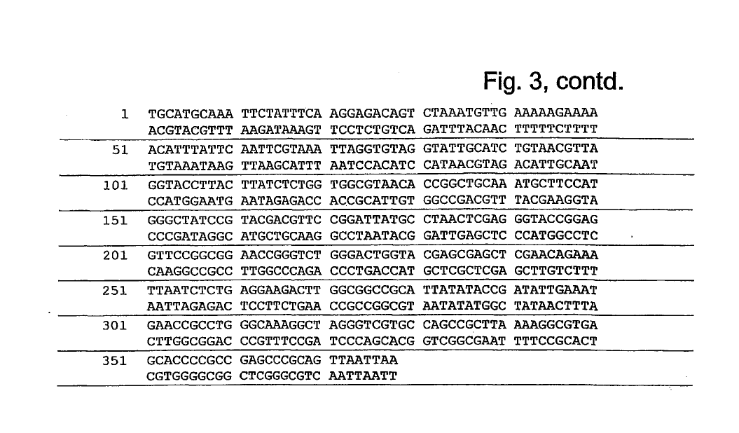

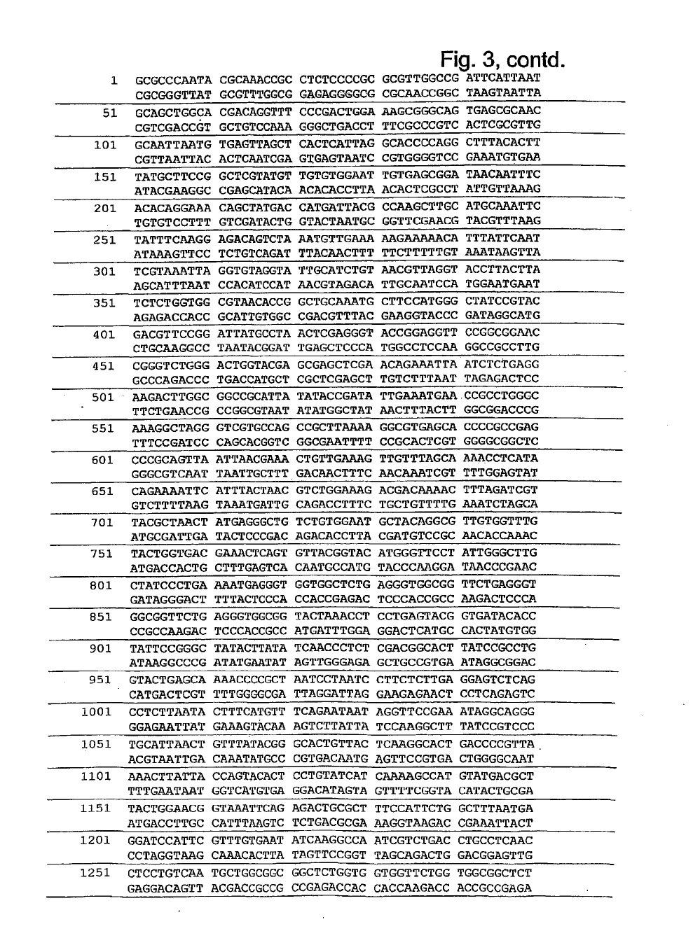

The phagemid PDV UO3 is the basis vector for generating a library of binding molecules consisting of variable heavy chain 3 domains. A nucleic acid sequence of the phagemid PDV UO3 is given in FIG. 1. Instead of gVIIIp protein in the PDV UO3 vector gIIIp can also be used. The core of the soluble VH3 domain is given in FIG. 2. The dots indicate places, representing CDR1 and CDR2 in an unaltered VH domain, where through varying the amino acid sequence, VH domains of various binding specificities can be obtained. The place marked “CDR3” in the figure, also indicates a place where through varying amino acids, VH domains comprising various binding specificities can be obtained. Of course said CDR3 regions may vary in size, at least according to the natural VH3 size variation in CDR3. By varying the amino acid sequence in the CDR regions it is possible to generate VH3 domains with varying specificities. The solubility of sVH3 versus an unmodified VH3 is due to mutations in framework 2 and framework 3, said mutations leading to a change in the hydrophobicity of the VH3 domain such that the hydrophilicity of the mutated VH3 domain increases. The solubility of sVH3 allows the generation of a phage comprising a binding molecule consisting of a VH domain in the absence of a light chain. Libraries of binding specificities based on sVH3 domains can be generated by methods known in the art as long as the basic amino-acid sequence given in FIG. 2 is used. Other amino-acid sequences then given in FIG. 2 can also be used provided that they result in a sufficiently soluble VH3 domain. A person skilled in the art can arrive at the library by for instance chosen primers with at least partial overlap and building an ever larger part of the sVH3 domain by consecutively amplifying resulting product with a further partially overlapping primer. The CDR3 domain being located at the extreme end of the VH domain requires attention in the amplification procedure. Preferably, one or more (partially overlapping) primers are used that result in a restriction site being present at the extreme end of the amplified product such that the resulting sVH3 library can easily be cloned into PDV UO3. A preferred combination of enzymes to clone the library into PDV UO3 is NcoI and XhoI, wherein NcoI is located near the leader in PDV UO3 that is fused to the start of the sVH3 domain. The resulting phagemids are electroporated into E. coli TG1 or XL1-blueTEN. The bacteria are plated onto suitable culture plates that include 5% glucose. The next day the resulting colonies are collected and stored. Several of these collections are inoculated in liquid medium and helper phages. After 1 night at degrees 30 C the phages are harvested. The resulting phages are selected for the appropriate target and amplified using said bacteria. The amplified phages were sequenced and shown to be as expected.

Generation of a structural protein capable of supporting proper VH3 function.

The phagemid PDV UO2 is the basis vector for generating a library of binding molecules consisting of variable heavy chain 3 domains further comprising a structural protein (SP) capable of supporting VH3 function. (SP does not comprise intrinsic antigen binding capacity). The sequence of a first SP (SPI) is obtained by shortening the binding loops of CDR1 and CDR2 in the light chain VΘ3 such that the binding properties are destroyed but the heavy chain supporting function of the light chain is essentially left intact. This is achieved by deleting amino acid from CDR1 and CDR2 such that these CDRs do not contain antigen binding capacity. In this Example, the 4 amino acids representing amino acid 28-31 are omitted from CDR1. These amino acids represent the most variable region in the CDR1 region of Vκ1 (012). From CDR2, 3 amino acids, representing amino acid 53-55 in Vκ1 (O12) are omitted. Vκ1 CDR3 is replaced by a VSV-tag. The VSV-tag used contains the amino acid sequence YTDIEMNRLGK. A nucleic acid encoding SP1 was generated synthetically using assembly PCR and the correctness of the nucleic acid sequence was verified by sequencing. The nucleic acid contains a NotI site and a SacI site such that cloning of SP1 into PDV UO2 does not disrupt the reading frame of the gIII protein. The NotI site is located near the putative N-terminal part of SP1.

SP2 was generated based on VK3 (A27) by omitting the 5 amino acids representing amino acid 28-31A are omitted from CDR1. These amino acids represent the most variable region in the CDR1 region of Vκ3 (A27). From CDR2, 3 amino acids, representing amino acid 53-55 in Vκ3 (A27) are omitted. The CDR3 of Vκ3 (A27) is replaced by a VSV-tag. The VSV-tag used contains the amino acid sequence YTDIEMNRLGK. A nucleic acid encoding SP2 was generated synthetically using assembly PCR and the correctness of the nucleic acid sequence was verified by sequencing. The nucleic acid contains a NotI site and a SacI site such that cloning of SP2 into PDV UO2 does not disrupt the reading frame of the gIII protein. The NotI site is located near the putative N-terminal part of SP2.

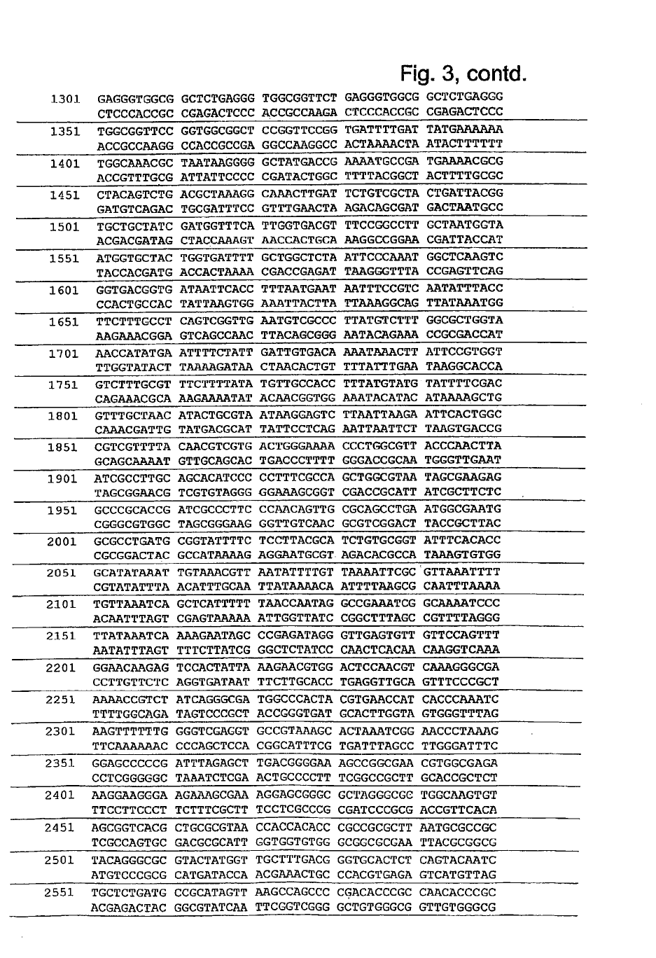

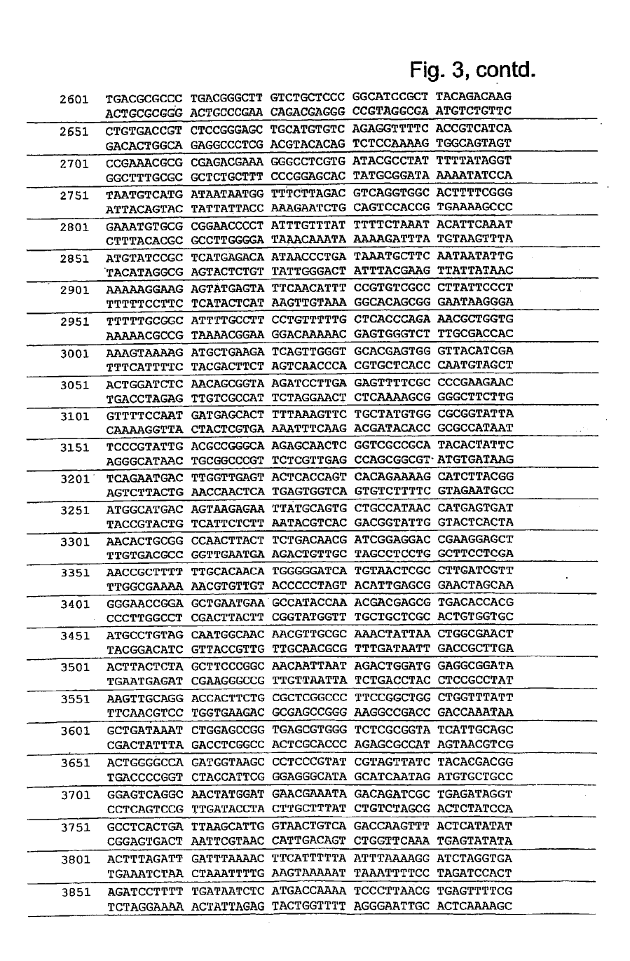

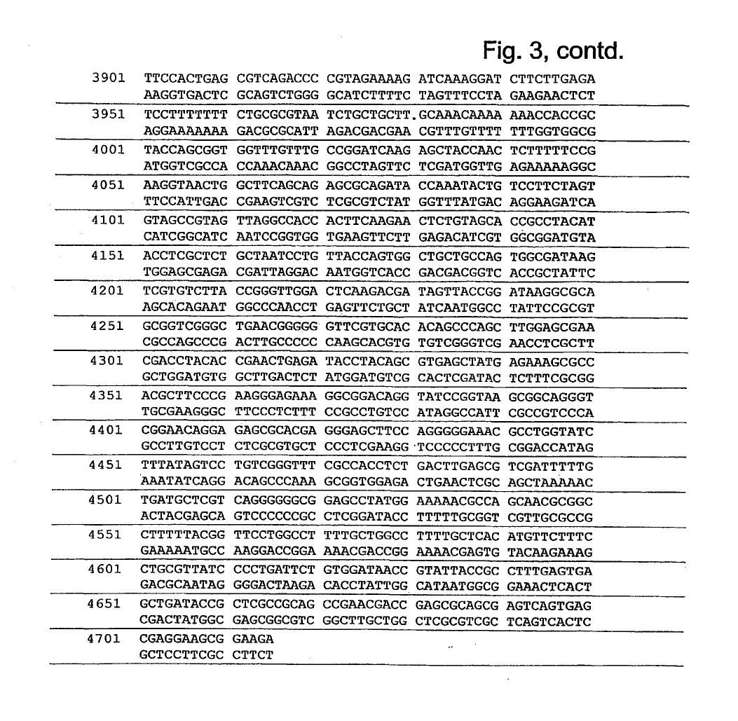

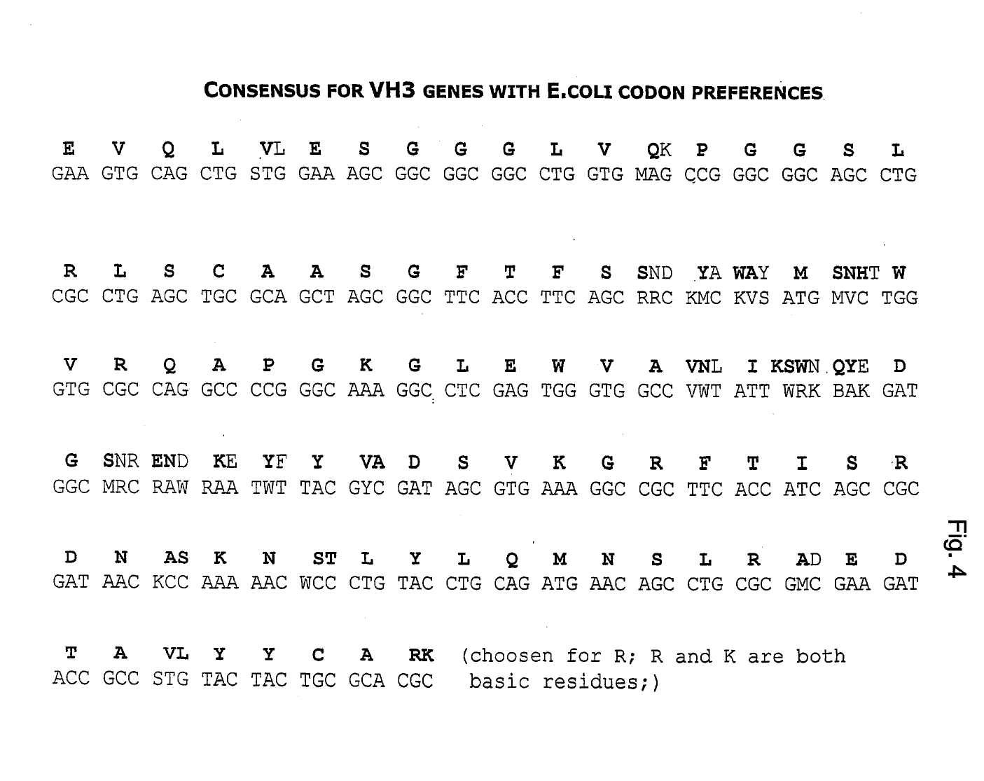

A VH3 framework and CDR1 and CDR2 randomized region used in this example is depicted in FIG. 4. The nucleic acid sequence encoding this VH3 framework is also given in FIG. 4. This nucleic sequence is optimized for codon usage in both E. coli and human cells. Table 1 depicts nucleic acid sequences that are optimized for codon usage in E. coli and human cells. The nucleic acid sequences encoding the framework are flanked by restriction sites NcoI and XhoI such that the reading frame of the gIII protein is left intact. The framework is cloned into PDV UO2 using the sites indicated. The resulting phagemids containing either SP1 together with the framework or SP2 together with the frame work are electropprated into E. coli TG1 or XL1-blueTEN. The bacteria are plated onto suitable culture plates that include 5% glucose. The next day the resulting colonies are collected and stored. Several of these collections are inoculated in liquid medium and helper phages. After 1 night at 30 degrees C., the phages are harvested. The resulting phages are selected for the appropriate target and amplified using said bacteria. The amplified phages were sequenced and shown to be as expected.

### REFERENCES

1 Berek, C., & Milstein, C. 1987. Mutation drift and repertoire shift in the maturation of the immune response. Immunol. Rev. 96:23.

2 Winter, G. & Milstein, C. 1991. Man-made antibodies. Nature. 349:293.

3 Vaughan, T. J., Osbourn, J. K., & Tempest, P. R. 1998. Human antibodies by design. Nat. Biotechnol. 16,535.

4 Winter, G., Griffiths, A. D., Hawkins, R. E., & Hoogenboom, H. R. 1994. Making antibodies by phage display technology. Annu. Rev. Immunol. 12:433.

5 Burton, D. R., & Barbas, C. F. 1994. Human antibodies from combinatorial libraries. Adv. Immunol. 57:191.

6 Hoogenboom, H. R. 1994. Designing and optimizing library selection strategies for generating high-affinity antibodies. Trends in Biotechnol. 15:62.

7 Marks, J. D., Griffiths, A. D., Malmqvist, M., Clackson, T., Bye, J. M., & Winter, G. 1992. Bypassing immunisation: high affinity human antibodies by chain shuffling. Bio/Technology. 10:779.

8 Clackson, T., Hoogenboom, H. R., Griffiths, A. D., & Winter, G. 1991. Making antibody fragments using phage display libraries. Nature., 352:624.

9 Hawkins, R. E., Russel, S. J., & Winter. G. 1992. Selection of phage antibodies by binding affinity: mimicking affinity maturation. J. Mol. Biol. 226:889.

10 Low, N.M., Holliger, P. H., & Winter, G. 1996. Mimicking somatic hypermutation: affinity maturation of antibodies displayed on bacteriophage using a bacterial mutator strain. J. Mol. Biol. 260,359.

11 Barba's, C.F., Hu, D., Dunlop, N., Sawyer, L., Cababa, D., Hendry, R. M., Nara, P. L., & Burton, D. R. 1994. In vitro evolution of a neutralizing human antibody to human immunodeficiency virus type 1 to enhance affinity and broaden strain cross-reactivity. Proc. Natl. Acad. Sci. USA. 91:3809.

12 Yang, W. -P., Green, K., Pinz-Sweeney, S., Briones, A. T., Burton, D. R., & Barbas, C. F. 1995. CDR walking mutagenesis for the affinity maturation of a potent human ant-HIV-1 antibody into the picomolar range. J. Mol. Biol. 254:392.

13 Balint, R. F., & Larrick, J. W. 1993. Antibody engineering by parsimonious mutagenesis. Gene. 137:109.

TABLE 1
CODON USAGE IN E. COLI AND H. SAPIENS

| Aminoacid | | Preferential | Alternative |
|---|---|---|---|
| Classic | Modern | codon | codons |
| Ala | A | GCC | GCT GCA |
| Cys | C | TGC | TGT |
| Asp | D | GAT | GAC |
| Glu | E | GAA | GAG |
| Phe | F | TTC | TTT |
| Gly | G | GGC | |
| His | H | CAC | CAT |
| Ile | I | ATC | ATT |
| Lys | K | AAA | AAG |
| Leu | L | CTG | |
| Met | M | ATG | |
| Asn | N | AAC | AAT |
| Pro | P | * | |
| Gln | Q | CAG | |
| Arg | R | CGC | |
| Ser | S | AGC | AGT TCC TCT |
| Thr | T | ACC | |
| Val | V | GTG | GTC |
| Trp | W | TGG | |
| Tyr | Y | TAC | TAT |
| *The codon usage in E. coli and H. sapiens does not correspond. The use of CCG is strongly preffered in E. coli while the proline codon in H. sapiens is strongly biased for CCC. The codon advised for the Phage Display Technique is CCG as the E. coli forms the basis of the selection. After maturation of the CDR-regions the proline codons might be replaced by CCC. This way both E.coli and human cell-lines may optimal synthesize | | | |
| # the desired single chain or other antibody products. | | | |

What is claimed is:

1. A process for producing a human monoclonal antibody, said method comprising:
providing a library of binding molecules, the binding domain of which consists essentially of human heavy chain variable fragments in a functional format,
selecting from said library of binding molecules at least one heavy chain variable fragment having a desired binding affinity,
inserting a nucleic acid encoding said heavy chain variable fragment having a desired binding affinity into a nucleic acid encoding the complementary part of at least a heavy chain of a human monoclonal antibody, and
allowing for expression of the resulting heavy chain and for assembly of said heavy chain with a desired light chain, thus producing a human monoclonal antibody.

2. The process of claim 1 wherein said heavy chain variable fragment having a desired binding affinity is in a functional format through fusion to a structural protein designed for that purpose.

3. The process of claim 1, wherein at least one sequence of said heavy chain variable fragment relevant only for association with a light chain is removed.

4. The process of claim 1, wherein the complementary part of the heavy chain is derived from VH3, VH4 or VH1.

5. The process of claim 1, wherein the light chain is derived from a member of a Vkappa1, Vkappa3 and Vlambda3 gene family.

6. Human monoclonal antibody produced by the process of claim 1.

7. Human monoclonal antibody produced by the process of claim 2.

8. Human monoclonal antibody produced by the process of claim 3.

9. Human monoclonal antibody produced by the process of claim 4.

10. Human monoclonal antibody produced by the process of claim 5.

11. A method for producing a structural amino acid sequence or a nucleic acid sequence encoding such an amino acid sequence for keeping a human heavy chain variable fragment in a functional format upon expression of a nucleic acid encoding such a fragment in a fusion with a nucleic acid encoding a protein expressed associated with the surface of a phage particle, said method comprising:
fusing a nucleic acid sequence encoding a possible structural amino acid sequence to a nucleic acid which is a fusion of a human heavy chain variable fragment with a known binding affinity and said nucleic acid encoding a protein expressed associated with the surface of a phage particle, and
expressing said nucleic acid in the context of a suitable phage expression system and selecting fusions which expose the desired binding affinity.

12. A proteinaceous substance or a nucleic acid encoding it, which substance is capable of keeping a heavy chain variable fragment in a functional conformation, produced by a method according to claim 11.

13. A method for making a library of binding molecules, said method comprising:
cloning a number of randomized nucleic acids derived from a heavy chain variable fragment in functional alignment with a nucleic acid encoding the proteinaceous substance of claim 12, and
providing the resulting nucleic acid in functional alignment with a nucleic acid encoding a protein expressed associated with the surface of a phage particle and expressing the resulting nucleic acids comprising said heavy chain variable fragment, said proteinaceous substance encoding acid and said surface protein encoding nucleic acid in the context of a suitable phage expression system, thus producing said library.

14. A phage display library obtainable by the method according to claim 13.
